(19) United States
(12) Patent Application Publication
(10) Pub. No.: US 2025/0000459 A1
(43) Pub. Date: Jan. 2, 2025

(54) Detecting Low Ejection Fraction using Photoplethysmography (PPG)

(71) Applicant: Google LLC, Mountain View, CA (US)

(72) Inventors: Ming-Zher Poh, Belmont, MA (US); John Weston Hughes, Palo Alto, CA (US); Paolo Di Achille, Medford, MA (US); Shun Liao, Cambridge, MA (US)

(21) Appl. No.: 18/343,465

(22) Filed: Jun. 28, 2023

(51) Int. Cl. A61B 5/00; A61B 5/024; A61B 5/318

(52) U.S. Cl. CPC A61B 5/7275; A61B 5/02416; A61B 5/318; A61B 5/681

(57) ABSTRACT

A computer-implemented method for detecting a cardiac dysfunction in a user includes obtaining, from a sensor, photoplethysmogram (PPG) signals indicative of a cardiac rhythm of the user. The computer-implemented method further includes processing, in a computing device, the PPG signals to generate a cardiac dysfunction prediction for the user based, at least in part, on the PPG signals. The computer-implemented method further includes providing, via an annunciator, the cardiac dysfunction prediction for the user as an output.

### FIELD

Example aspects of the present disclosure generally relate to wearable devices.

### BACKGROUND

A wearable computing device can be worn, for instance, on a user's wrist. The wearable computing device can include a plurality of sensors such as, for example biometric sensors. The biometric sensors can obtain data indicative of the user's physiological state. For instance, such data can include data representative of, for example, a cardiac rhythm of the user.

### SUMMARY

Aspects and advantages of embodiments of the present disclosure will be set forth in part in the following description, or can be learned from the description, or can be learned through practice of the embodiments.

In one aspect, a computer-implemented method for detecting a cardiac dysfunction in a user is provided. The method includes obtaining, from a sensor, photoplethysmogram (PPG) signals indicative of a cardiac rhythm of the user. The method further includes processing, in a computing device, the PPG signals to generate a cardiac dysfunction prediction for the user based, at least in part, on the PPG signals. The method further includes providing, via an annunciator, the cardiac dysfunction prediction for the user as an output.

In some implementations, processing the PPG signals to generate a cardiac dysfunction prediction for the user may include processing, via one or more machine-learned models, the PPG signals to generate the cardiac dysfunction prediction for the user.

In some implementations, the method may further include obtaining, via the computing device, electrocardiogram (ECG) signals indicative of the cardiac rhythm of the user. The method may further include processing, via the one or more machine-learned models, the PPG signals and the ECG signals to generate the cardiac dysfunction prediction for the user. Furthermore, in some implementations, the ECG signals may be obtained via a single-lead electrocardiogram. In some implementations, the one or more machine-learned models may be further configured to process demographic data relating to the user to generate the cardiac dysfunction prediction for the user.

In some implementations, the one or more machine-learned models may be trained with PPG data, ECG data, and demographic data. Furthermore, in some implementations, the one or more machine-learned models may include a deep learning model.

In some implementations, the cardiac dysfunction prediction for the user may include one or more predicted probabilities that the user is experiencing the cardiac dysfunction.

In some implementations, obtaining the PPG signals may include obtaining, via one or more biometric sensors of a wearable computing device, PPG signals indicative of the cardiac rhythm of the user. Furthermore, in some implementations, the one or more biometric sensors may include one or more PPG sensors.

In some implementations, obtaining the PPG signals may include obtaining, via one or more pulse oximeters, PPG signals indicative of the cardiac rhythm of the user.

In some implementations, obtaining the PPG signals may include obtaining, via one or more cameras of a mobile computing device, PPG signals indicative of the cardiac rhythm of the user.

In some implementations, the method may further include surfacing, via the computing device, a notification to the user indicative of the cardiac dysfunction prediction via a user interface of a wearable computing device. Furthermore, in some implementations, surfacing a notification to the user indicative of the cardiac dysfunction prediction may further include surfacing, via the computing device, a recommendation to the user to wear an ECG monitor in response to generating the cardiac dysfunction prediction.

In some implementations, the cardiac dysfunction may be left ventricular systolic dysfunction (LVSD).

In another aspect, a wearable computing device is provided. The wearable computing device includes one or more sensors configured to obtain photoplethysmogram (PPG) data of a user wearing the wearable computing device. The wearable computing device further includes one or more computing devices. The one or more computing devices are configured to obtain PPG signals, from the sensors, indicative of a cardiac rhythm of the user. The one or more computing devices are further configured to provide the PPG signals to one or more remote computing devices. The one or more computing devices are further configured to receive, from the one or more remote computing devices, an indication of a cardiac dysfunction of the user.

In some implementations, the wearable computing device may further include electrocardiogram (ECG) sensors configured to obtain ECG data indicative of the cardiac rhythm of the user.

In some implementations, the one or more computing devices may include a machine-learned model. Furthermore, the wearable computing device may be configured to determine the cardiac dysfunction of the user via the machine-learned model based, at least in part, on the PPG data of the user.

In some implementations, the one or more computing devices may be further configured to surface, via a user interface of the wearable computing device, a notification to the user indicative of the cardiac dysfunction in response to receiving the indication of the cardiac dysfunction of the user.

In another aspect, a computing system for detection of a cardiac dysfunction in a user is provided. The computing system includes one or more processors and one or more non-transitory computer-readable media. The one or more non-transitory computer-readable media collectively store one or more machine-learned cardiac dysfunction detection models and instructions that, when executed by the one or more processors, cause the computing system to perform operations. The one or more machine-learned cardiac dysfunction detection models are configured to provide cardiac dysfunction predictions based, at least in part, on photoplethysmogram (PPG) recordings. Furthermore, the operations include obtaining PPG signals indicative of a cardiac rhythm of the user. The operations further include processing the PPG signals with the one or more machine-learned cardiac dysfunction detection models to generate a cardiac dysfunction prediction for the user based, at least in part, on the PPG signals. The operations further include providing the cardiac dysfunction prediction for the user as an output.

Other aspects of the present disclosure are directed to various systems, apparatuses, non-transitory computer-readable media, user interfaces, and electronic devices.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1A depicts an embodiment of a wearable computing device according to example embodiments of the present disclosure;

FIG. 1B depicts a rear perspective view of the wearable computing device of the wearable computing device of FIG. 1A according to example embodiments of the present disclosure;

FIG. 2 depicts an embodiment of a mobile smart phone device according to example embodiments of the present disclosure;

FIG. 3 depicts an embodiment of a mobile tablet device according to example embodiments of the present disclosure;

FIG. 4A depicts a block diagram of components of a wearable computing device according to example embodiments of the present disclosure;

FIG. 4B depicts a block diagram of the example machine-learned cardiac dysfunction detection model of FIG. 4A according to example embodiments of the present disclosure;

FIG. 5 depicts a flow chart diagram of an example method for implementing a machine-learned cardiac dysfunction detection model according to example embodiments of the present disclosure; and

FIG. 6 depicts a computing system according to example embodiments of the present disclosure;

FIG. 7A depicts a block diagram of an example computing system for implementing cardiac dysfunction detection models according to example embodiments of the present disclosure;

FIG. 7B depicts a block diagram of an example computing system for implementing cardiac dysfunction detection models according to example embodiments of the present disclosure; and

FIG. 7C depicts a block diagram of an example computing system for implementing cardiac dysfunction detection models according to example embodiments of the present disclosure.

Repeat use of reference characters in the present specification and drawings is intended to represent the same and/or analogous features or elements of the present invention.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Example aspects of the present disclosure generally relate to systems and methods that leverage machine learning for detection of cardiac dysfunctions such as, for example, left ventricular systolic dysfunction (LVSD), based on sensor data obtained from one or more biometric sensors. In particular, a computing system can include and use one or more machine-learned cardiac dysfunction detection models to provide cardiac dysfunction predictions for a user based on sensor data obtained by one or more biometric sensors such as, e.g., photoplethysmogram (PPG) sensors. Specifically, in some example embodiments, a computing system can obtain one or more PPG signals (e.g., PPG recordings) that are indicative of a cardiac rhythm of a user. The computing system can process the one or more PPG signals with the one or more machine-learned cardiac dysfunction detection models to generate a cardiac dysfunction prediction for the user relative to one or more cardiac dysfunctions such as, e.g., LVSD. The computing system can provide the cardiac dysfunction prediction for the user as an output (e.g., to the user, to a medical profession, to an electronic medical record system, and/or various other systems or processes).

The one or more PPG signals can be obtained in a variety of ways. For instance, in some embodiments, a PPG recording can be obtained using a smartphone by having the user place their finger over the camera. Additionally and/or alternatively, the PPG recording can be obtained by a pulse oximeter. Additionally and/or alternatively, the PPG recording can be obtained by a wearable device (e.g., a smartwatch) having one or more PPG sensors.

For instance, example aspects of the present disclosure are directed to a wearable computing device that can be worn, for example, on a user's wrist. The wearable computing device includes a PPG sensor that is configured to generate a PPG signal indicative of a biometric (e.g., heart rate) of the user. The PPG sensor includes an emitter that includes one or more light sources (e.g., light emitting diodes (LEDs)) configured to emit light toward a body part of the user when the wearable computing device is worn by the user. The PPG sensor further includes one or more detectors (e.g., photodiodes) configured to receive a reflection of the light emitted toward the body part. It should be understood that the PPG signal is the reflection of the light.

As discussed herein, the machine-learned cardiac dysfunction detection model is configured to detect a cardiac dysfunction such as, e.g., LVSD, based on the one or more PPG signals. Asymptomatic LVSD is present in approximately two percent of the population. Additionally, asymptomatic LVSD is present in approximately nine percent of the elderly population. Moderate or greater LVSD is defined as having an ejection fraction of less than forty percent. Furthermore, LVSD confers approximately a five-fold increase in the risk of clinical heart failure and approximately a two-fold increase in all-cause mortality.

Early detection of LVSD is crucial. If detected early, treatments (e.g., cardiac rehab, ACE inhibitors, beta blockers, etc.) are effective in preventing the progression to symptomatic heart failure which, in turn, reduces mortality. However, LVSD often goes undetected and, thus, undiagnosed. At present, the best-studied test for screening for LVSD is an invasive blood test which tests for B-type natriuretic peptide (BNP) levels. Such tests, however, have an area under the receiver operating characteristic curve (AUC) (i.e., a measure of test accuracy) of approximately 0.6-0.7.

Accordingly, example aspects of the present disclosure provide systems and methods for detecting LVSD in a user using the machine-learned cardiac dysfunction detection model. According to example embodiments of the present disclosure, the machine-learned cardiac dysfunction detection model can be configured to process one or more PPG signals and, as a result, output a cardiac dysfunction prediction indicative of LVSD in a user. Furthermore, by training with and leveraging ECG data and demographic data (in addition to PPG data), the systems and methods disclosed herein can provide an AUC of, approximately, 0.86. Furthermore, example aspects of the present disclosure can surface a notification to the user based on the cardiac dysfunction prediction which, in turn, can lead to increased early detection and decreased mortality.

Furthermore, in contrast to current medical and experimental detection approaches which require invasive testing (e.g., blood tests) and/or electrocardiogram (ECG) recordings, example embodiments of the present disclosure are able to detect LVSD in the user directly from PPG signals, which was heretofore thought to be impossible. Moreover, accessibility to screening is significantly increased, including among the at-risk and/or elderly population, given the widespread ownership of smartphones and smartwatches. Furthermore, in the case of wearable devices, the PPG-based screening and detection approach provided herein enables passive monitoring of ejection fraction without the need for active spot-checks by a user (e.g., ECG monitoring). Even further, by combining the biometric data from the PPG sensor(s) and ECG(s) with demographic data, the accuracy and performance of the disclosed systems and methods is significantly improved. In this way, false positive results and false negative results can be greatly reduced, which is important for consumer-facing products.

In addition to the above-described effects and benefits, example aspects of the present disclosure provide numerous additional technical effects and benefits. More particularly, the systems and methods of the present disclosure provide improved techniques, for detecting cardiac dysfunctions based on PPG data indicative of a cardiac rhythm of a user, using one or more machine-learned cardiac dysfunction detection models. In addition, the information provided by the machine-learned cardiac dysfunction detection model can improve the accuracy of diagnoses and user health-related outcomes. As such, the disclosed system can significantly reduce the cost and time needed to provide diagnostic information and can result in improved medical care for users. Furthermore, the systems and methods described herein may provide resulting improvements to computing technology tasked with monitoring and detecting cardiac dysfunctions in users. Improvements in the speed and accuracy of determining and detecting cardiac dysfunctions can directly improve operational speeds for computing systems. For instance, by improving diagnostic accuracy (e.g., by reducing false positives and false negatives), the number of duplicative diagnostic operations can be reduced-thereby reducing processing and storage requirements for the computing systems. Hence, the reduced processing and storage requirements ultimately result in more efficient resource use for the computing system. In this way, valuable computing resources within a computing system that would have otherwise been needed for such tasks can be reserved for other tasks (e.g., extracting and processing additional cardiac information from biometric data, increasing storage capacity, etc.).

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (e.g., “A or B” is intended to mean “A or B or both”). The term “at least one of” in the context of, e.g., “at least one of A, B, and C” refers to only A, only B, only C, or any combination of A, B, and C. In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration.” In addition, references to “an embodiment” or “one embodiment” do not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

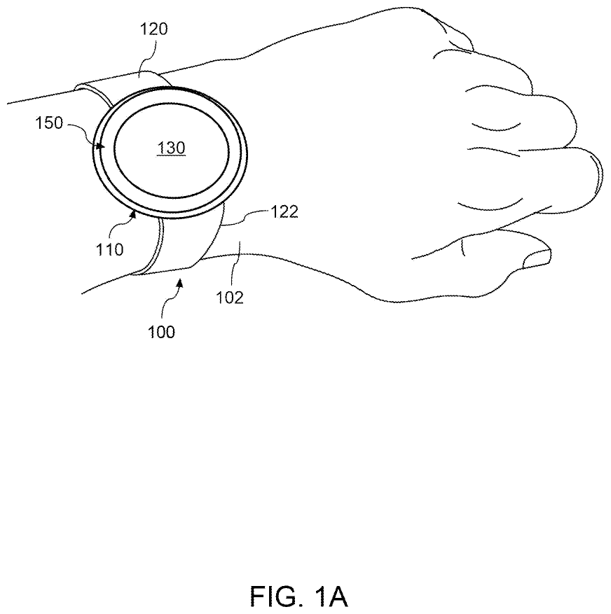

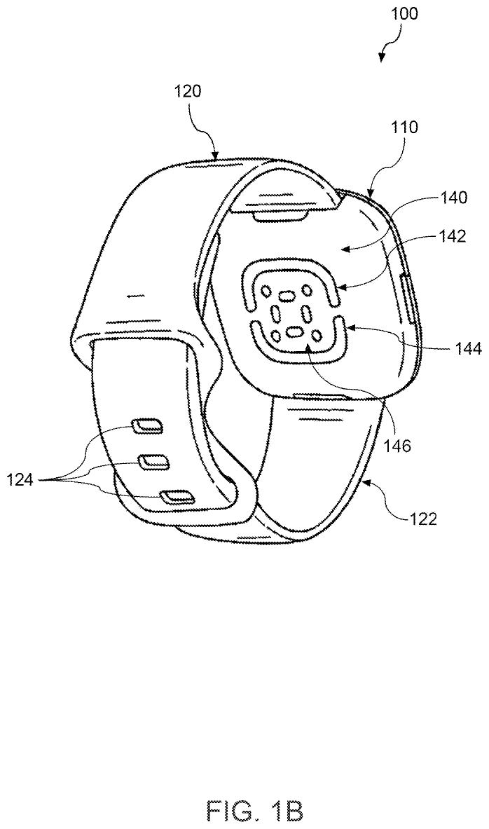

Referring now to the Figures, FIG. 1A through FIG. 1B illustrate a wearable computing device 100 according to some implementations of the present disclosure. As shown, the wearable computing device 100 can be worn, for instance, on an arm 102 (e.g., wrist) of a user. The wearable computing device 100 can include a housing 110. The housing 110 can define a cavity (e.g., internal volume) (not shown) in which one or more electronic components (e.g., disposed on printed circuit boards) are disposed. For instance, the wearable computing device 100 can include a printed circuit board (e.g., flexible printed circuit board) (not shown) disposed within the cavity. Furthermore, one or more electronic components can be disposed on the printed circuit board. The wearable computing device 100 can further include a battery (not shown) that is disposed within the cavity defined by the housing.

As shown, the wearable computing device 100 can include a first band 120 coupled to the housing 110 at a first location and a second band 122 coupled to the housing 110 at a second location. The first band 120 and the second band 122 can be coupled to one another at a third location (not shown) to secure the housing 110 to the arm 102 of the user. For instance, the first band 120 can include a buckle or clasp (not shown). Additionally, the second band 122 can define a plurality of apertures 124 spaced apart from one another along a length of the second band 122. In such embodiments, a prong of the buckle associated with the first band 120 can extend through one of the plurality of openings defined by the second band 122 to couple the first band 120 to the second band 122.

It should be appreciated that the first band 120 can be coupled to the second band 122 using any suitable type of fastener. For instance, in some embodiments, the first band 120 and the second band 122 can include a magnet (not shown). In such embodiments, the first band 120 and the second band 122 can be magnetically coupled to one another to secure the housing 110 to the arm 102 of the user.

The wearable computing device 100 can include a display 130 (FIG. 1A) configured to display content (e.g., time, date, biometric, notifications, etc.) for viewing by the user. The display 130 can include a plurality of pixels. For instance, in some embodiments, the display 130 can include an organic light-emitting diode (OLED) display. It should be understood, however, that the display 130 can include any suitable type of display.

In some implementations, the wearable computing device 100 can include an electrocardiogram (ECG) sensor 140. For instance, the ECG sensor 140 can include a first electrode 142 (FIG. 1B) and a second electrode 144 (FIG. 1B). It should be understood that the wearable computing device 100 can include more or fewer electrodes. As shown, the first electrode 142 and the second electrode 144 are positioned with respective apertures (e.g., cutouts) defined by the housing 110. Furthermore, the first electrode 142 and the second electrode 144 can each contact (e.g., touch) the wrist of the user. In this manner, the first electrode 142 and the second electrode 144 can be used to measure one or more biometrics (e.g., electrodermal activity, electrocardiogram) of the user.

The wearable computing device 100 can include an optical sensor package 146 (FIG. 1B) for measuring data for one or more biometrics of the user of the wearable computing device 100. As shown, in some implementations, the ECG sensor 140 can be disposed around optical sensor package 146 on a wrist-side of the housing 110. The optical sensor package 146 can be configured to obtain, for example, optical PPG measurements. The optical sensor package 146 can include one or more emitters and one or more detectors. The optical sensor package 146 can further include a lens extending across the optical sensor package 146 such that light emitted from the one or more emitters can exit the housing 110 of the wearable computing device 100 via the lens. In this manner, the emitter(s) and detector(s) of the optical sensor package 146 can be disposed such that the lens can also cover and/or protect the emitter(s) and detector(s) of the optical sensor package 146.

Some PPG technologies rely on detecting light at a single spatial location, adding signals taken from two or more spatial locations, or an algorithmic combination thereof. Both of these approaches result in a single spatial measurement from which the heart rate (HR) estimate (or other physiological metrics) can be determined. In some embodiments, a PPG device employs a single light source coupled to a single detector (i.e., a single light path). Alternatively, a PPG device may employ multiple light sources coupled to a single detector or multiple detectors (i.e., two or more light paths). In other embodiments, a PPG device employs multiple detectors coupled to a single light source or multiple light sources (i.e., two or more light paths). In some cases, the light source(s) may be configured to emit one or more of green, red, infrared (IR) light, as well as any other suitable wavelengths in the spectrum (such as long IR for metabolic monitoring). For example, a PPG device may employ a single light source and two or more light detectors each configured to detect a specific wavelength or wavelength range. In some cases, each detector is configured to detect a different wavelength or wavelength range from one another. In other cases, two or more detectors are configured to detect the same wavelength or wavelength range. In yet another case, one or more detectors configured to detect a specific wavelength or wavelength range different from one or more other detectors). In embodiments employing multiple light paths, the PPG device may determine an average of the signals resulting from the multiple light paths before determining an HR estimate or other physiological metrics.

The wearable computing device 100 can include a display cover 150 positioned on the housing 110 such that the display cover 150 is positioned on top of the display 130. In this manner, the display cover 150 can protect the display 130 from being damaged (e.g., scratched or cracked). In some embodiments, the wearable computing device 100 can include a seal (not shown) positioned between the housing 110 and the display cover 150. For instance, a first surface of the seal can contact the housing 110 and a second surface of the seal can contact the display cover 150. In this manner, the seal between the housing 110 and the display cover 150 can prevent a liquid (e.g., water) from entering the cavity defined by the housing 110.

It should be understood that the display cover 150 (FIG. 1A) can be optically transparent so that the user can view information being displayed on the display 130 (FIG. 1A). For instance, in some embodiments, the display cover 150 can include a glass material. It should be understood, however, that the display cover 150 can include any suitable optically transparent material.

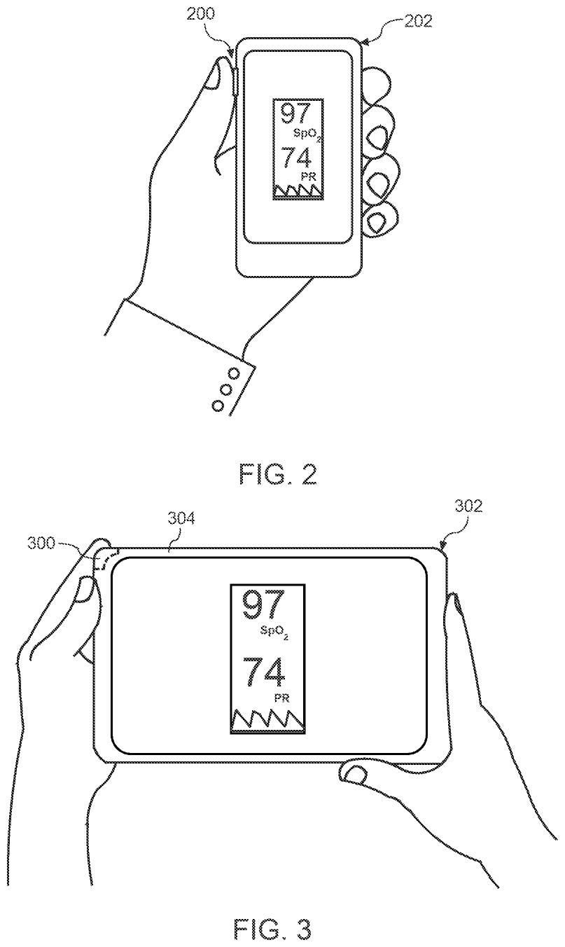

FIG. 2 depicts a diagram of an example optical sensor package 200 implemented in a mobile smart phone device 202 according to example embodiments of the present disclosure. As shown, optical sensor package 200 can be implemented as a button disposed on a side surface of the mobile smart phone device 202. Furthermore, in some implementations, the optical sensor package 200 can be coupled to a camera system of the smart phone device 202. As such, camera flash technology of the camera system of the smart phone device 202 can be utilized to illuminate an area on the user using the light output by the camera flash to capture the user's physiological data using the optical sensor package 200 coupled to the camera system. Furthermore, the optical sensor package 200 depicted in FIG. 2 can include the same structure, components, attributes, and/or functionality as that of optical sensor package 146 illustrated in, and discussed above, with reference to FIG. 1B.

FIG. 3 depicts a diagram of an example optical sensor package 300 implemented in a mobile tablet device 302 according to example embodiments of the present disclosure. As shown, optical sensor package 300 can be disposed (e.g., embedded) in a slotted section (not shown) of an external shell 304 of mobile tablet device 302 (e.g., at an edge, corner, or bezel portion of external shell 304). Additionally and/or alternatively, the optical sensor package 300 can be implemented as a button disposed on a side surface of the mobile tablet device 302. In some implementations, the optical sensor package 300 can be coupled to a camera system of the mobile tablet device 302. As such, camera flash technology of the camera system of the mobile tablet device 302 can be utilized to illuminate an area on the user using the light output by the camera flash to capture the user's physiological data using the optical sensor package 300 coupled to the camera system. Furthermore, the optical sensor package 300 depicted in FIG. 3 can include the same structure, components, attributes, and/or functionality as that of optical sensor package 146 illustrated in, and discussed above, with reference to FIG. 1B.

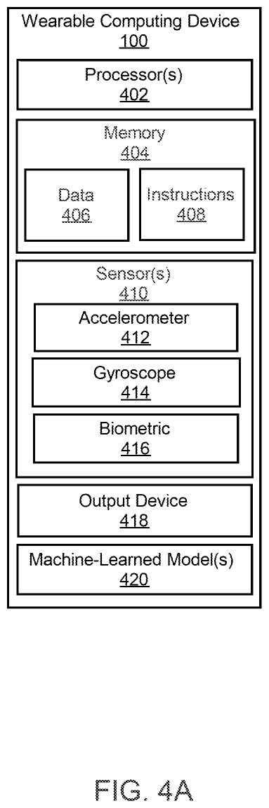

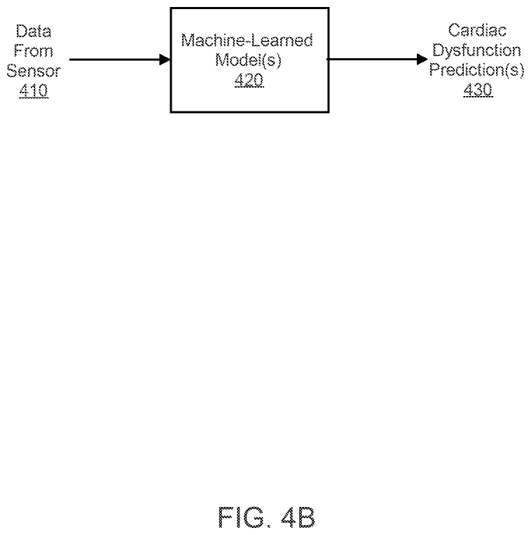

Referring now to FIG. 4A, a block diagram of components of a wearable computing device 100 is provided according to some embodiments of the present disclosure. It should be understood that the wearable computing device 100 can be employed within the system 600 discussed below with reference to FIG. 6.

As shown, the wearable computing device 100 can include one or more processors 402. The one or more processors 402 can include any suitable processing device (e.g., a processor core, a microprocessor, an application specific integrated circuit (AISC), a field programmable gate array (FPGA), a microcontroller, etc.). The wearable computing device 100 can further include a memory 404. The memory 404 can include one or more non-transitory computer-readable storage media, such as random-access memory (RAM), read-only memory (ROM), electronically erasable programmable ready-only memory (EEPROM), erasable programmable read-only memory (EPROM), flash memory devices, and combinations thereof. The memory 404 can store data 406 and instructions 408 that, when executed by the one or more processors 402, cause the one or more processors 402 to perform operations disclosed herein.

The wearable computing device 100 can include a plurality of sensors 410. For instance, in some embodiments, the plurality of sensors 410 can include an accelerometer 412 (e.g., a multi-axis accelerometer) and a gyroscope 414. In this manner, the accelerometer 412, the gyroscope 414, or both can obtain motion data (e.g., acceleration, angular velocity) indicative of movement of the user.

In some embodiments, the plurality of sensors 410 can include one or more biometric sensors 416. For instance, in some embodiments, the one or more biometric sensors 416 can include one or more photoplethysmogram (PPG) sensors (e.g., optical sensor package 146) configured to obtain PPG data (e.g., PPG recordings) indicative of a cardiac rhythm of the user. Furthermore, the one or more biometric sensors 416 can include an electrocardiogram (ECG) (e.g., ECG sensor 140) configured to obtain ECG data (e.g., ECG signals) indicative of the cardiac rhythm of the user. Alternatively, or additionally, the sensors 410 can include a temperature sensor (not shown) configured to detect a body temperature of the user wearing the wearable computing device 100.

In some embodiments, the wearable computing device 100 can include one or more output devices 418. For instance, the one or more output devices 418 can include a display screen (e.g., display 130). In this manner, the wearable computing device 100 can display content (e.g., notifications) that can be viewed by the user. Alternatively, or additionally, the one or more output devices 418 can include one or more speakers. In this manner, the wearable computing device 100 can emit audible noises (e.g., alarm, voice automated message, etc.) for the user. As will be discussed below, the wearable computing device 100 can be configured to assess the cardiac rhythm of the user to determine whether the user is experiencing and/or will experience a cardiac dysfunction such as, e.g., left ventricular systolic dysfunction (LVSD).

In some embodiments, the one or more processors 402 can be communicatively coupled to the plurality of sensors 410. For instance, the one or more processors 402 can be communicatively coupled to the plurality of sensors 410 via a data interface (e.g., data bus). In this manner, the one or more processors 402 can obtain data from the plurality of sensors 410. In some embodiments, the one or more processors 402 can determine a cardiac dysfunction event of the user based, at least in part, on the data obtained from the one or more sensors 410.

In some embodiments, biometric data obtained from the one or more biometric sensors 416 of the wearable computing device 100 can indicate whether the wearable computing device 100 is currently being worn by the user. For instance, in some embodiments, the biometric data obtained from the one or more biometric sensors 416 can indicate the wearable computing device 100 is not being worn (e.g., off-wrist) by the user. In such embodiments, the one or more processors 402 can be configured to disable cardiac rhythm-related data collection functionality while the biometric data obtained from the one or more biometric sensors 416 indicates the wearable computing device 100 is not being worn by the user. In this manner, erroneous data from the one or more biometric sensors 416 can be ignored. It should be understood that the one or more processors 402 can be configured to enable cardiac rhythm-related data collection functionality when the biometric data obtained from the one or more biometric sensors 416 indicates the wearable computing device 100 is being worn (e.g., on-wrist) by the user.

In some embodiments, the wearable computing device 100 can include one or more machine-learned models 420 (e.g., machine-learned model 720, machine-learned model 740). For instance, in some embodiments, the one or more machine-learned models 420 can be stored in the memory 404 of the wearable computing device 100. In alternative embodiments, the one or more machine-learned models 420 can be stored in the memory of one or more devices that are remote relative to the wearable computing device 100. For instance, in some embodiments, the one or more machine-learned models 420 can be stored in memory of the mobile computing device 610 (FIG. 6) that is communicatively coupled with the wearable computing device 100 via a network 620 (FIG. 6). Alternatively, or additionally, the one or more machine-learned models 420 can be stored on one or more servers (not shown) that are communicatively coupled with the wearable computing device 100 via the network 620. As will now be discussed, the one or more machine-learned models 420 (e.g., machine-learned cardiac dysfunction detection models) can be configured to generate a cardiac dysfunction prediction 430 based, at least in part, on data obtained from the one or more sensors 410.

FIG. 4B depicts a block diagram of an example machine-learned cardiac dysfunction detection model 420 according to example embodiments of the present disclosure. In some embodiments, the machine-learned cardiac dysfunction detection model 420 is configured to provide cardiac dysfunction predictions 430 based, at least in part, on sensor data from the one or more sensors 410. For instance, in some embodiments, the machine-learned cardiac dysfunction model 420 can be configured to provide the one or more cardiac dysfunction predictions 430 based, at least in part, on photoplethysmogram (PPG) data obtained from one or more PPG sensors and/or one or more electrocardiogram (ECG) signals obtained from one or more ECG sensors. It should be noted that “machine-learned cardiac dysfunction detection model,” “cardiac dysfunction detection model,” and “machine-learned model” can be used interchangeably.

As will be discussed in greater detail below, the one or more machine-learned models 420 can be trained or configured to provide a cardiac dysfunction prediction 430 relative to one or more cardiac dysfunctions (e.g., left ventricular systolic dysfunction (LVSD)) based on sensor data indicative of a cardiac rhythm of a user from the one or more sensors 410. For instance, in some embodiments, data from one or more biometric sensors 416 (e.g., one or more PPG sensors) can be provided as an input to the one or more machine-learned models 420. In some embodiments, the one or more machine-learned models 420 can be trained with PPG data and ECG data. Furthermore, in some embodiments, the one or more machine-learned models 420 can be trained with demographic data.

The one or more machine-learned models 420 can process the data from the one or more sensors 410 to generate an output indicative of the cardiac dysfunction of the user (e.g., cardiac dysfunction prediction 430) based, at least in part, on the data obtained from the one or more sensors 410. In some embodiments, the output of the one or more machine-learned models 420 can be a single numerical value that can be used to indicate whether a cardiac dysfunction event is occurring and/or will occur in the future. In alternative embodiments, the output of the one or more machine-learned models 420 can include a plurality of outputs. For instance, in some embodiments, the cardiac dysfunction prediction 430 can include one or more probabilities that the user is experiencing the cardiac dysfunction. As one example, the cardiac dysfunction prediction 430 can indicate that the user is, with 78% probability, currently experiencing one or more cardiac dysfunctions.

In some embodiments, the one or more machine-learned models 420 can be configured to process one or more PPG signals from one or more PPG sensors and ECG data from one or more ECGs to generate the cardiac dysfunction prediction 430. Furthermore, as noted above, the one or more machine-learned models 420 can be configured to process demographic data relating to the user to generate the cardiac dysfunction prediction 430.

It should be understood that the one or more machine-learned models 420 can include any suitable type of machine-learned model. For instance, the one or more machine-learned models 420 can include, without limitation, a convolutional neural network, a decision tree, a Bayesian network, a support vector machine, a K-means cluster, or any other suitable type of machine-learned model and/or deep learning models.

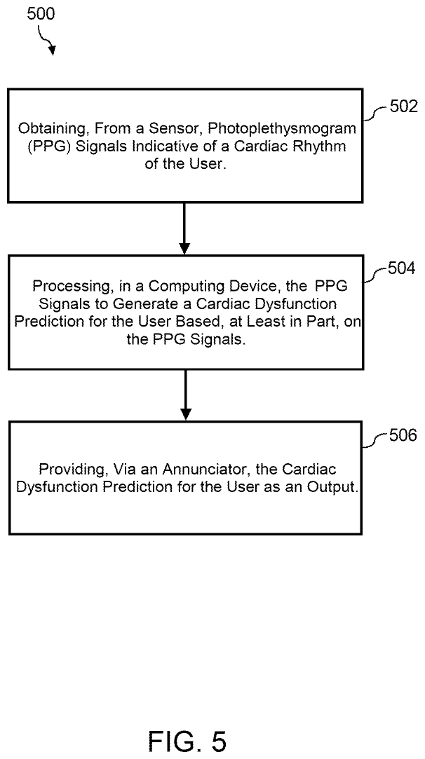

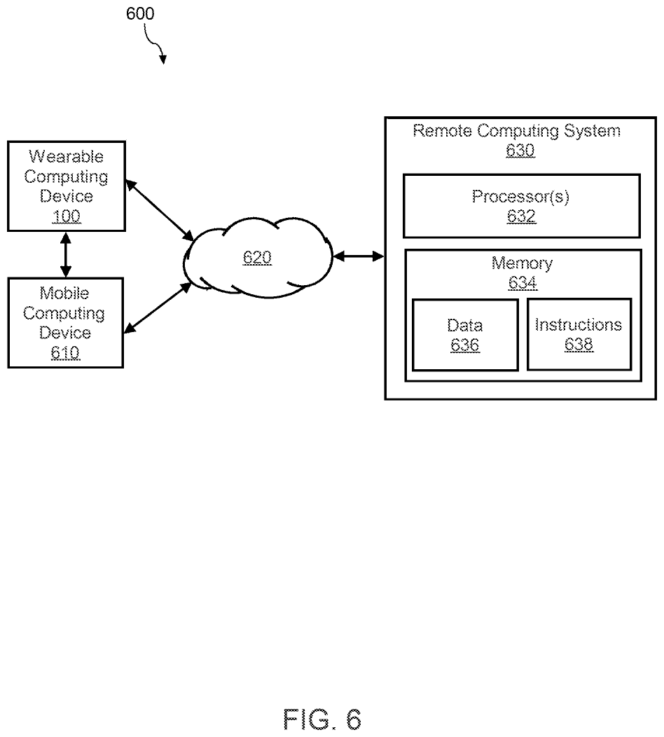

Referring now to FIG. 5, a flow diagram of an example method 500 for detecting a cardiac dysfunction in a user is provided according to example embodiments of the present disclosure. The method 500 may be implemented using, for instance, the wearable computing device 100 discussed above with reference to FIGS. 1A-1B, the mobile smart phone device 202 discussed above with reference to FIG. 2, and/or the mobile tablet device 302 discussed above with reference to FIG. 3. Alternatively, the method 500 may be implemented by a computing device (e.g., server, smartphone, etc.) that is communicatively coupled to the wearable computing device 100. It should be understood that, in some embodiments, some steps of the method 500 may be implemented locally on the wearable computing device 100, mobile smart phone device 202, and/or mobile tablet device 302, whereas other steps of the method 500 may be implemented by a computing device that is remote from the wearable computing device 100, the mobile smart phone device 202, and/or the mobile tablet device 302, and is communicatively coupled to the wearable computing device 100, the mobile smart phone device 202, and/or the mobile table device 302 via one or more wireless networks.

FIG. 5 depicts steps performed in a particular order for purposes of illustration and discussion. Those of ordinary skill in the art, using the disclosures provided herein, will understand that various steps of any of the methods described herein can be omitted, expanded, performed simultaneously, rearranged, and/or modified in various ways without deviating from the scope of the present disclosure. Furthermore, various steps (not illustrated) can be performed without deviating from the scope of the present disclosure. Additionally, the method 500 is generally discussed with reference to the wearable computing device 100 discussed above with reference to FIGS. 1A-1B, the mobile smart phone device 202 discussed above with reference to FIG. 2, the mobile tablet device 302 discussed above with reference to FIG. 3, the plurality of sensors 410 discussed above with reference to FIGS. 4A-4B, and the machine-learned model 420 discussed above with reference to FIGS. 4A-4B. However, it should be understood that aspects of the present method 500 can find application with any suitable computing device, sensor, and/or machine-learned model.

The method 500 can include, at (502), obtaining, from a sensor, photoplethysmogram (PPG) signals indicative of a cardiac rhythm of the user. For instance, one or more sensors (e.g., one or more biometric sensors) of a wearable computing device can be configured to obtain biometric data of a user wearing the wearable computing device. The biometric data can include data indicative of a cardiac rhythm of the user such as, e.g., PPG signals and/or electrocardiogram (ECG) data. In some embodiments, the one or more biometric sensors can include one or more PPG sensors (e.g., optical sensor package 146). Additionally and/or alternatively, the one or more PPG signals indicative of the cardiac rhythm of the user can be obtained by one or more pulse oximeters. Additionally and/or alternatively, the one or more PPG signals indicative of the cardiac rhythm of the user can be obtained by one or more cameras from a mobile computing device such as, e.g., a cell phone (e.g., mobile smart phone device 202) or a tablet (e.g., mobile tablet device 302). Those of ordinary skill in the art will understand that the PPG signals can be obtained in any suitable manner without deviating from the scope of the present disclosure.

Additionally and/or alternatively, the method 500 can include obtaining, via the computing device, ECG signals indicative of the cardiac rhythm of the user. In some embodiments, the ECG signals can be obtained by ECGs of the wearable computing device (e.g., one or more single-lead ECGs) configured to obtain ECG data indicative of the cardiac rhythm of the user (e.g., ECG sensor 140). Those having ordinary skill in the art will appreciate that the one or more ECG signals can be obtained by any suitable ECG configured to obtain ECG data indicative of a cardiac rhythm of the user without deviating from the scope of the present disclosure.

The method 500 can include, at (504), processing, in a computing device, the PPG signals to generate a cardiac dysfunction prediction for the user based, at least in part, on the PPG signals. More particularly, the method 500 can include processing, via one or more machine-learned models, the PPG signals to generate the cardiac dysfunction prediction for the user. For instance, the one or more machine-learned models can include one or more machine-learned cardiac dysfunction models configured to provide cardiac dysfunction predictions based, at least in part, on biometric data such as, e.g., PPG recordings and/or ECG data. In some embodiments, the one or more machine-learned cardiac dysfunction models can be deep learning model(s).

In some embodiments, the PPG signals can be processed at one or more remote computing devices via the one or more machine-learned models. For instance, one or more computing devices of the wearable computing device can be configured to provide the PPG signals obtained at (502) to the one or more remote computing devices. Furthermore, the one or more machine-learned models can be configured to generate an output indicative of a cardiac dysfunction of the user such as, e.g., left ventricular systolic dysfunction (LVSD).

Additionally and/or alternatively, the method 500 can include processing, via the one or more machine-learned models, the PPG signals and the ECG signals to generate the cardiac dysfunction prediction for the user. In some embodiments, the one or more machine-learned models are further configured to process demographic data relating to the user to generate the cardiac dysfunction prediction for the user. Furthermore, the one or more machine-learned models can be trained with PPG data, ECG data, and/or demographic data.

The method 500 can include, at (506), providing, via an annunciator, the cardiac dysfunction prediction for the user as an output. More particularly, the one or more machine-learned models can be configured to output the cardiac dysfunction prediction for the user based, at least in part, on the one or more PPG signals obtained at (502). In some embodiments, the cardiac dysfunction prediction for the user can include one or more predicted probabilities that the user is experiencing the cardiac dysfunction. However, those of ordinary skill in the art will appreciate that the cardiac dysfunction prediction can be any suitable format without deviating from the scope of the present disclosure.

The annunciator can include, for example, a display of the wearable computing device (e.g., wearable computing device 100), a display of a mobile smart phone device (e.g., mobile smart phone device 202), or a display of a mobile tablet device (e.g., mobile tablet device 302). Additionally and/or alternatively, the annunciator can include a speaker of the wearable computing device (e.g., wearable computing device 100), a speaker of the mobile smart phone device (e.g., mobile smart phone device 202), or a speaker of the mobile tablet device (e.g., mobile tablet device 302). Additionally and/or alternatively, the annunciator can include a haptic device (e.g., a vibrating device) of the wearable computing device (e.g., wearable computing device 100), a haptic device of the mobile smart phone device (e.g., mobile smart phone device 202), or a haptic device of the mobile tablet device (e.g., mobile tablet device 302).

For instance, the method 500 can include surfacing, via the computing device, a notification to the user indicative of the cardiac dysfunction prediction via a user interface (e.g., display 130) of the wearable computing device (e.g., wearable computing device 100). In some embodiments, the computing devices of the wearable computing device can be configured to receive an indication of a cardiac dysfunction of the user from the one or more remote computing devices. The indication of the cardiac dysfunction of the user can be based, at least in part, on the PPG signals and ECG signals obtained by the one or more sensors. For example, in response to generating the cardiac dysfunction prediction at (506), the computing device can be configured to surface the notification to the user via a display device of the wearable computing device. In some embodiments, the notification can include a recommendation for the user to wear an ECG monitor.

Additionally and/or alternatively, the method 500 can include surfacing a notification to the user indicative of the cardiac dysfunction prediction via a user interface of a mobile smart phone device (e.g., mobile smart phone device 202) and/or a mobile tablet device (e.g., mobile tablet device 302). Additionally and/or alternatively, the computing device can be configured to surface the notification to a medical professional and/or medical institution (e.g., primary care physician, clinic, etc.). Moreover, it should be appreciated that the notification may be provided in any other suitable way or combination of ways, for instance by haptic feedback, by audio-feedback, and/or the like.

FIG. 6 depicts an example computing system 600 according to example embodiments of the present disclosure. The computing system 600 can be used, for instance, to implement the method 500 of FIG. 5 or other aspects of any of the methods described herein. The computing system 600 includes the wearable computing device 100 discussed above with reference to FIGS. 1A-1B and a remote computing system 630. The wearable computing device 100 can be communicatively coupled to the remote computing system 630 over a network 620. Furthermore, the computing system 600 can include one or more machine-learned models (not shown) such as, e.g., machine-learned models 420.

In some embodiments, the wearable computing device 100 can communicate the data to mobile computing device 610. In such embodiments, the wearable computing device 100 can communicate the data to the mobile computing device 610 and then the mobile computing device 610 can communicate the data over the network 620 to the remote computing system 630. In alternative embodiments, the wearable computing device 100 can bypass the mobile computing device 610 and instead communicate the data directly to the remote computing system 630 via the network 620.

The remote computing system 630 includes one or more processors 632 and a memory 634. The one or more processors 632 can be any suitable processing device (e.g., a processor core, a microprocessor, an ASIC, an FPGA, a controller, a microcontroller, etc.) and can be one processor or a plurality of processors that are operatively connected. The memory 634 can include one or more non-transitory computer-readable storage medium(s), such as RAM, ROM, EEPROM, EPROM, flash memory devices, magnetic disks, etc., and combinations thereof. The memory 634 can store data 636 and instructions 638 which are executed by the processor 632 to cause the remote computing system 630 to perform operations, such as any of the operations described herein. For instance, in some embodiments, the memory 634 of the remote computing system 630 can be configured to store the one or more machine-learned models 420 discussed above with reference to FIGS. 1-5. In this manner, the data obtained from one or more sensors 410 (e.g., accelerometer, gyroscope, biometric, etc.) onboard the wearable computing device 100 and indicative of a cardiac rhythm of the user can be communicated to the remote computing system 630 and provided as an input to the one or more machine-learned models 420 stored in the memory 634 thereof. The one or more machine-learned models 420 can be configured to process the data and output cardiac dysfunction predictions based, at least in part, on data obtained from the one or more sensors 410. Furthermore, the cardiac dysfunction prediction can be communicated over the network 620 to the wearable computing device 100.

In some embodiments, the remote computing system 630 includes or is otherwise implemented by one or more computing devices. In instances in which the remote computing system 630 includes plural server computing devices, such server computing devices can operate according to sequential computing architectures, parallel computing architectures, or some combination thereof.

The network 620 can be any type of communications network, such as a local area network (e.g., intranet), wide area network (e.g., Internet), or some combination thereof and can include any number of wired or wireless links. In general, communication over the network 620 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

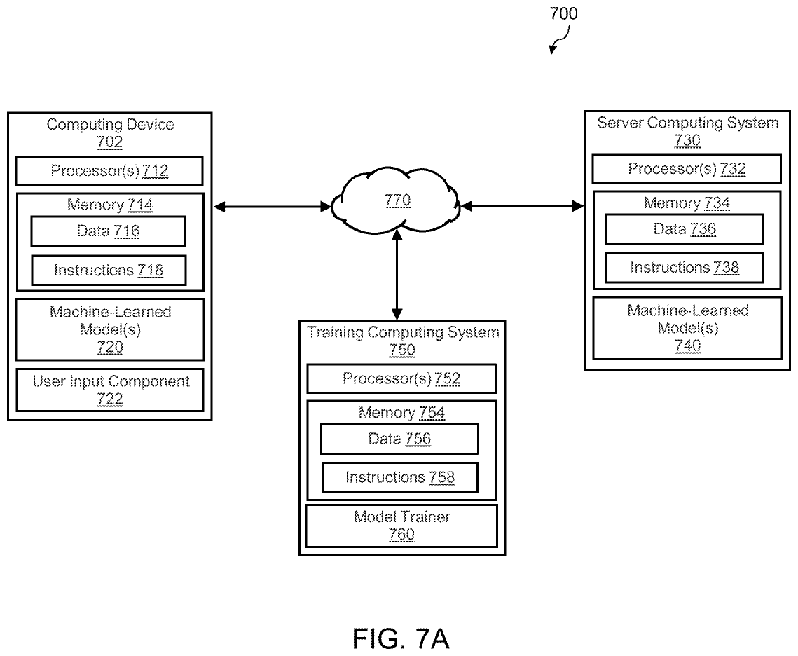

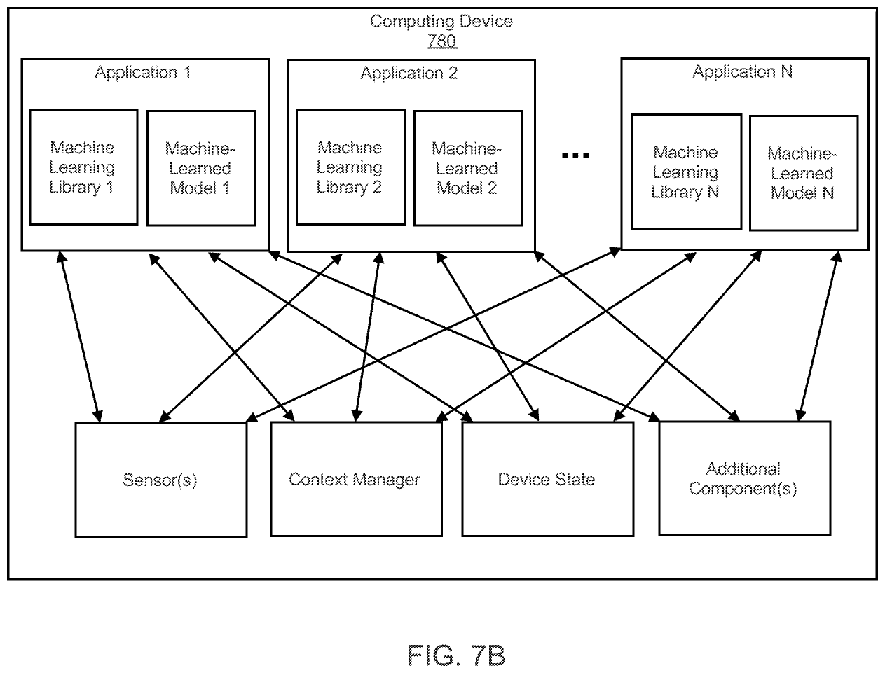

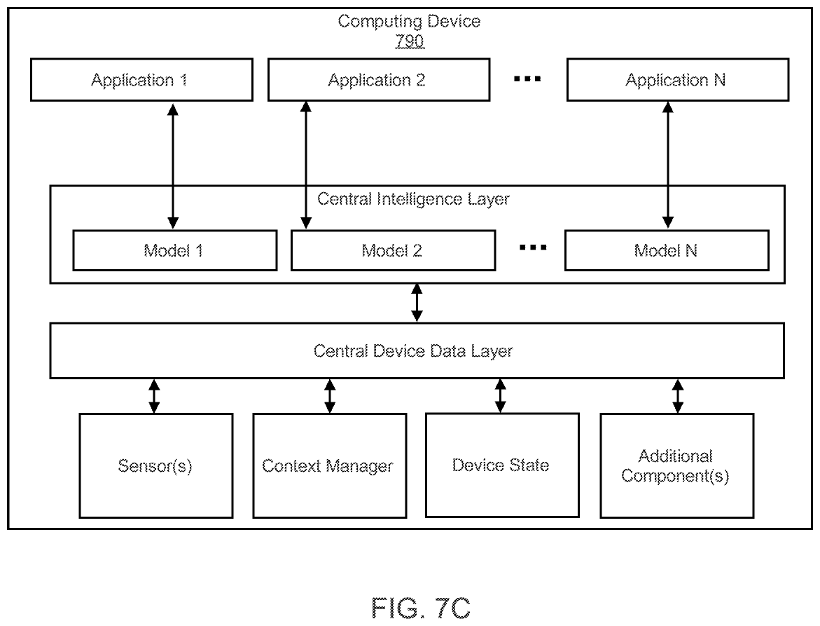

FIG. 7A depicts a block diagram of an example computing system 700 that can perform according to example embodiments of the present disclosure. The system 700 includes a computing device 702, a server computing system 730, and a training computing system 750 that are communicatively coupled over a network 770.

The computing device 702 can be any type of computing device, such as, for example, a personal computing device (e.g., laptop or desktop), a mobile computing device (e.g., smartphone or tablet), a gaming console or controller, a wearable computing device, an embedded computing device, or any other type of computing device. In some embodiments, the computing device 702 can be a client computing device. The computing device 702 can include one or more processors 712 and a memory 714. The one or more processors 712 can be any suitable processing device (e.g., a processor core, a microprocessor, an ASIC, an FPGA, a controller, a microcontroller, etc.) and can be one processor or a plurality of processors that are operatively connected. The memory 714 can include one or more non-transitory computer-readable storage media, such as RAM, ROM, EEPROM, EPROM, flash memory devices, magnetic disks, etc., and combinations thereof. The memory 714 can store data 716 and instructions 718 which are executed by the processor 712 to cause the user computing device 702 to perform operations (e.g., to perform operations implementing input data structures and self-consistency output sampling according to example embodiments of the present disclosure, etc.).

In some implementations, the user computing device 702 can store or include one or more machine-learned models 720. For example, the machine-learned models 720 can be or can otherwise include various machine-learned models such as neural networks (e.g., deep neural networks) or other types of machine-learned models, including non-linear models or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models).

In some implementations, one or more machine-learned models 720 can be received from the server computing system 730 over network 770, stored in the computing device memory 714, and used or otherwise implemented by the one or more processors 712. In some implementations, the computing device 702 can implement multiple parallel instances of a machine-learned model 720.

Additionally, or alternatively, one or more machine-learned models 740 can be included in or otherwise stored and implemented by the server computing system 730 that communicates with the computing device 702 according to a client-server relationship.

The machine-learned models described in this specification may be used in a variety of tasks, applications, and/or use cases. Although described throughout with respect to example implementations for applications in medical domains, it is to be understood that the techniques described herein may be used for other tasks in various technological fields.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g., one or more images or videos), the output comprises compressed visual data, and the task is a visual data compression task. In another example, the task may comprise generating an embedding for input data (e.g., input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some embodiments, the machine-learned models 740 can be implemented by the server computing system 730 as a portion of a web service (e.g., remote machine-learned model hosting service, such as an online interface for performing machine-learned model operations over a network on remote servers 730). For instance, the server computing system 730 can communicate with the computing device 702 over a local intranet or internet connection. For instance, the computing device 702 can be a workstation or endpoint in communication with the server computing system 730, with implementation of the model 740 on the server computing system 730 being remotely performed and an output provided (e.g., cast, streamed, etc.) to the computing device 702. Thus, one or more models 720 can be stored and implemented at the user computing device 702 or one or more models 740 can be stored and implemented at the server computing system 730.

The computing device 702 can also include one or more input components that receive user input. For example, a user input component can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 730 can include one or more processors 732 and a memory 734. The one or more processors 732 can be any suitable processing device (e.g., a processor core, a microprocessor, an ASIC, an FPGA, a controller, a microcontroller, etc.) and can be one processor or a plurality of processors that are operatively connected. The memory 734 can include one or more non-transitory computer-readable storage media, such as RAM, ROM, EEPROM, EPROM, flash memory devices, magnetic disks, etc., and combinations thereof. The memory 734 can store data 736 and instructions 738 which are executed by the processor 732 to cause the server computing system 730 to perform operations (e.g., to perform operations implementing input data structures and self-consistency output sampling according to example embodiments of the present disclosure, etc.).

In some implementations, the server computing system 730 includes or is otherwise implemented by one or more server computing devices. In instances in which the server computing system 730 includes plural server computing devices, such server computing devices can operate according to sequential computing architectures, parallel computing architectures, or some combination thereof.

As described above, the server computing system 730 can store or otherwise include one or more machine-learned models 740. For example, the models 740 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models).

The computing device 702 or the server computing system 730 can train example embodiments of a machine-learned model (e.g., including models 720 or 740) using a pretraining pipeline (e.g., an unsupervised pipeline, a semi-supervised pipeline, etc.). In some embodiments, the computing device 702 or the server computing system 730 can train example embodiments of a machine-learned model (e.g., including models 720 or 740) using a pretraining pipeline by interaction with the training computing system 750. In some embodiments, the training computing system 750 can be communicatively coupled over the network 770. The training computing system 750 can be separate from the server computing system 730 or can be a portion of the server computing system 730.

The training computing system 750 can include one or more processors 752 and a memory 754. The one or more processors 752 can be any suitable processing device (e.g., a processor core, a microprocessor, an ASIC, an FPGA, a controller, a microcontroller, etc.) and can be one processor or a plurality of processors that are operatively connected. The memory 754 can include one or more non-transitory computer-readable storage media, such as RAM, ROM, EEPROM, EPROM, flash memory devices, magnetic disks, etc., and combinations thereof. The memory 754 can store data 756 and instructions 758 which are executed by the processor 752 to cause the training computing system 750 to perform operations (e.g., to perform operations implementing input data structures and self-consistency output sampling according to example embodiments of the present disclosure, etc.). In some implementations, the training computing system 750 includes or is otherwise implemented by one or more server computing devices.

The model trainer 760 can include a pretraining pipeline for training machine-learned models using various objectives. Parameters of the image-processing model(s) can be trained, in some embodiments, using various training or learning techniques, such as, for example, backwards propagation of errors. For example, an objective or loss can be backpropagated through the pretraining pipeline(s) to update one or more parameters of the model(s) (e.g., based on a gradient of the loss function). Various determinations of loss can be used, such as mean squared error, likelihood loss, cross entropy loss, hinge loss, or various other loss functions. Gradient descent techniques can be used to iteratively update the parameters over a number of training iterations. In some implementations, performing backwards propagation of errors can include performing truncated backpropagation through time. The pretraining pipeline can perform a number of generalization techniques (e.g., weight decays, dropouts, etc.) to improve the generalization capability of the models being trained.

The model trainer 760 can include computer logic utilized to provide desired functionality. The model trainer 760 can be implemented in hardware, firmware, or software controlling a general-purpose processor. For example, in some implementations, the model trainer 760 includes program files stored on a storage device, loaded into a memory, and executed by one or more processors. In other implementations, the model trainer 760 includes one or more sets of computer-executable instructions that are stored in a tangible computer-readable storage medium such as RAM, hard disk, or optical or magnetic media.

The network 770 can be any type of communications network, such as a local area network (e.g., intranet), wide area network (e.g., Internet), or some combination thereof and can include any number of wired or wireless links. In general, communication over the network 770 can be carried via any type of wired or wireless connection, using a wide variety of communication protocols (e.g., TCP/IP, HTTP, SMTP, FTP), encodings or formats (e.g., HTML, XML), or protection schemes (e.g., VPN, secure HTTP, SSL).

FIG. 7A illustrates one example computing system that can be used to implement the present disclosure. Other computing systems can be used as well. For example, in some implementations, the computing device 702 can include the model trainer 760. In some implementations, the computing device 702 can implement the model trainer 760 to personalize the model(s) based on device-specific data.

FIG. 7B depicts a block diagram of an example computing device 780 that performs according to example embodiments of the present disclosure. The computing device 780 can be a user computing device or a server computing device. The computing device 780 can include a number of applications (e.g., applications 1 through N). Each application can contain its own machine learning library and machine-learned model(s). For example, each application can include a machine-learned model. Example applications include a text messaging application, an email application, a dictation application, a virtual keyboard application, a browser application, etc. As illustrated in FIG. 7B, each application can communicate with a number of other components of the computing device, such as, for example, one or more sensors, a context manager, a device state component, or additional components. In some implementations, each application can communicate with each device component using an API (e.g., a public API). In some implementations, the API used by each application is specific to that application.

FIG. 7C depicts a block diagram of an example computing device 790 that performs according to example embodiments of the present disclosure. The computing device 790 can be a user computing device or a server computing device. The computing device 790 can include a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer can include a number of machine-learned models. For example, as illustrated in FIG. 7C, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of the computing device 790.

The central intelligence layer can communicate with a central device data layer. The central device data layer can be a centralized repository of data for the computing device 790. As illustrated in FIG. 7C, the central device data layer can communicate with a number of other components of the computing device, such as, for example, one or more sensors, a context manager, a device state component, or additional components. In some implementations, the central device data layer can communicate with each device component using an API (e.g., a private API).

The technology discussed herein makes reference to sensors, servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, processes discussed herein can be implemented using a single device or component or multiple devices or components working in combination. Likewise, server processes discussed herein may be implemented using a single server or multiple servers working in combination. Databases and applications may be implemented on a single system or distributed across multiple systems. Distributed components may operate sequentially or in parallel.

While the present subject matter has been described in detail with respect to specific example embodiments thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

Aspects of the disclosure have been described in terms of illustrative embodiments thereof. Any and all features in the following claims can be combined or rearranged in any way possible, including combinations of claims not explicitly enumerated in combination together, as the example claim dependencies listed herein should not be read as limiting the scope of possible combinations of features disclosed herein. Accordingly, the scope of the present disclosure is by way of example rather than by way of limitation, and the subject disclosure does not preclude inclusion of such modifications, variations or additions to the present subject matter as would be readily apparent to one of ordinary skill in the art. Moreover, terms are described herein using lists of example elements joined by conjunctions such as “and,” “or,” “but,” etc. It should be understood that such conjunctions are provided for explanatory purposes only. Clauses and other sequences of items joined by a particular conjunction such as “or,” for example, can refer to “and/or,” “at least one of”, “any combination of” example elements listed therein, etc. Also, terms such as “based on” should be understood as “based at least in part on.”

What is claimed is:

1. A computer-implemented method for detecting a cardiac dysfunction in a user, the method comprising:
obtaining, from a sensor, photoplethysmogram (PPG) signals indicative of a cardiac rhythm of the user;
processing, in a computing device, the PPG signals to generate a cardiac dysfunction prediction for the user based, at least in part, on the PPG signals; and
providing, via an annunciator, the cardiac dysfunction prediction for the user as an output.

2. The computer-implemented method of claim 1, wherein processing the PPG signals to generate a cardiac dysfunction prediction for the user comprises:
processing, via one or more machine-learned models, the PPG signals to generate the cardiac dysfunction prediction for the user.

3. The computer-implemented method of claim 2, further comprising:
obtaining, via the computing device, electrocardiogram (ECG) signals indicative of the cardiac rhythm of the user;
processing, via the one or more machine-learned models, the PPG signals and the ECG signals to generate the cardiac dysfunction prediction for the user.

4. The computer-implemented method of claim 3, wherein the ECG signals are obtained via a single-lead electrocardiogram.

5. The computer-implemented method of claim 3, wherein the one or more machine-learned models are further configured to process demographic data relating to the user to generate the cardiac dysfunction prediction for the user.

6. The computer-implemented method of claim 2, wherein the one or more machine-learned models are trained with PPG data, ECG data, and demographic data.

7. The computer-implemented method of claim 2, wherein the one or more machine-learned models comprise a deep learning model.

8. The computer-implemented method of claim 1, wherein the cardiac dysfunction prediction for the user comprises one or more predicted probabilities that the user is experiencing the cardiac dysfunction.

9. The computer-implemented method of claim 1, wherein obtaining the PPG signals comprises:
obtaining, via one or more biometric sensors of a wearable computing device, PPG signals indicative of the cardiac rhythm of the user.

10. The computer-implemented method of claim 9, wherein the one or more biometric sensors comprise one or more PPG sensors.

11. The computer-implemented method of claim 1, wherein obtaining PPG signals comprises:
obtaining, via one or more pulse oximeters, PPG signals indicative of the cardiac rhythm of the user.

12. The computer-implemented method of claim 1, wherein obtaining PPG signals comprises:
obtaining, via one or more cameras of a mobile computing device, PPG signals indicative of the cardiac rhythm of the user.

13. The computer-implemented method of claim 1, further comprising:
surfacing, via the computing device, a notification to the user indicative of the cardiac dysfunction prediction via a user interface of a wearable computing device.

14. The computer-implemented method of claim 13, wherein surfacing a notification to the user indicative of the cardiac dysfunction prediction further comprises:
surfacing, via the computing device, a recommendation to the user to wear an ECG monitor in response to generating the cardiac dysfunction prediction.

15. The computer-implemented method of claim 1, wherein the cardiac dysfunction is left ventricular systolic dysfunction (LVSD).

16. A wearable computing device comprising:
one or more sensors configured to obtain photoplethysmogram (PPG) data of a user wearing the wearable computing device; and
one or more computing devices configured to:
obtain PPG signals, from the sensors, indicative of a cardiac rhythm of the user;
provide the PPG signals to one or more remote computing devices; and
receive, from the one or more remote computing devices, an indication of a cardiac dysfunction of the user.

17. The wearable computing device of claim 16, further including:
electrocardiogram (ECG) sensors configured to obtain ECG data indicative of the cardiac rhythm of the user.

18. The wearable computing device of claim 16, wherein:
the one or more computing devices comprise a machine-learned model; and
the wearable computing device is configured to determine the cardiac dysfunction of the user via the machine-learned model based, at least in part, on the PPG data of the user.

19. The wearable computing device of claim 16, wherein the one or more computing devices are further configured to:
surface, via a user interface of the wearable computing device, a notification to the user indicative of the cardiac dysfunction in response to receiving the indication of the cardiac dysfunction of the user.

20. A computing system for detection of a cardiac dysfunction in a user, the computing system comprising:
one or more processors; and
one or more non-transitory computer-readable media that collectively store:
one or more machine-learned cardiac dysfunction detection models configured to provide cardiac dysfunction predictions based, at least in part, on photoplethysmogram (PPG) recordings; and
instructions that, when executed by the one or more processors, cause the computing system to perform operations, the operations comprising:
obtaining PPG signals indicative of a cardiac rhythm of the user;
processing the PPG signals with the one or more machine-learned cardiac dysfunction detection models to generate a cardiac dysfunction prediction for the user based, at least in part, on the PPG signals; and
providing the cardiac dysfunction prediction for the user as an output.
